(19) United States
(12) Patent Application Publication
(10) Pub. No.: US 2019/0224192 A1
(43) Pub. Date: Jul. 25, 2019

(54) Stable Pharmaceutical Composition of Vortioxetine Hydrobromide

(71) Applicant: Cipla Limited, Mumbai (IN)

(72) Inventors: Geena MALHOTRA, Mumbai (IN); Dharmaraj Ramachandra RAO, Thane (West) (IN); Venkata Srinivas PULLELA, Bangalore (IN); Preeti RAUT, Mumbai (IN); Shrikant Suresh MUDGAL, Panvel (IN); Pratap Ramesh SAWANT, Panvel (IN); Jinesh CHAUHAN, Mumbai (IN); Nidhi BAGREE, Navi Mumbai (IN)

(21) Appl. No.: 16/329,078

(22) PCT Filed: Aug. 29, 2017
(86) PCT No.: PCT/GB2017/052521

§ 371 Date: Feb. 27, 2019

(30) Foreign Application Priority Data
Aug. 29, 2016 (IN) 201621029379

(51) Int. Cl. A61K 31/495; A61K 9/14; A61K 9/20

(52) U.S. Cl. CPC A61K 31/495; A61K 9/146; A61K 9/2031; A61K 9/2027; A61K 9/2095; A61K 9/2054

(57) ABSTRACT

The present invention relates to novel premixes of Vortioxetine, processes for the preparation of such premixes, pharmaceutical compositions comprising the same and their use in medicine.

### CROSS REFERENCE TO RELATED APPLICATIONS

This application is a filing under 35 U.S.C. 371 of International Application No. PCT/GB2017/052521 filed Aug. 29, 2017, entitled “Stable Pharmaceutical Composition of Vortioxetine Hydrobromide” which claims priority to Indian Patent Application No. 201621029379 filed Aug. 29, 2016, which applications are incorporated by reference herein in their entirety.

### FIELD OF INVENTION

The present invention relates to a Vortioxetine hydrobromide premix comprising Vortioxetine hydrobromide in crystalline form and a process for preparing the said premix. The present invention also relates to a pharmaceutical composition comprising crystalline Vortioxetine hydrobromide, a process for preparing such a composition and medical uses thereof. The invention further relates to a method of stabilizing a composition comprising Vortioxetine hydrobromide.

### BACKGROUND

Vortioxetine (1 42-(2,4-dimethyl-phenylsulfanyl)-phenyThpiperazine) belonging to a chemical class of psychotropics, the bis-aryl-sulfanyl amines, is structurally different from all currently known psychotropics. Vortioxetine (L.U-AA21004) was approved as hydrobromide salt in US, on Sep. 30, 2013 and is marketed by Takeda Pharms USA under the tradename Trintellix®, (earlier Brintellix®). Trintellix® is available in dosages equivalent to 5, 10, 15 and 20 mg of the free base, in the form of oral immediate release film-coated tablets.

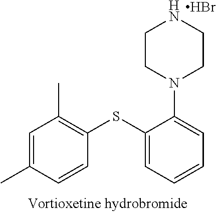 [SMILES: Br ; Cc1ccc(Sc2ccccc2N2CCNCC2)c(C)c1]

It is an oral antidepressant 5-HT transporter inhibitor. The mechanism of action is thought to be related to its enhancement of serotonergic activity in the Central Nervous System (CNS) through inhibition of the reuptake of serotonin (5-HT). It also has several other activities including 5-HT3 receptor antagonism and 5-HT1A receptor agonism. The contribution of these activities to Vortioxetine's antidepressant effect has not been established.

Trintellix® is indicated in the US for the treatment of major depressive disorder (MDD). In the EU, the drug is indicated for the treatment of adults with major depressive episodes. Development in other psychiatric indications is ongoing.

U.S. Pat. Nos. 7,144,884 and 8,476,279 disclose Phenyl-piperazine derivatives as serotonin reuptake inhibitors. While one of the many compounds claimed in the U.S. Pat. No. 7,144,884 is Vortioxetine, U.S. Pat. No. 8,476,279 specifically relates to Vortioxetine and its composition comprising a pharmaceutical carrier or diluent.

The marketed vortioxetine immediate-release film-coated tablets contain the hydrobromide salt of the drug in the crystalline beta form.

U.S. Pat. No. 8,722,684, discloses crystalline vortioxetine base and salts, including hemihydrate of vortioxetine hydrobromide as well as ethyl acetate solvate, crystalline vortioxetine hydrochloride and monohydrate of hydrochloride and crystalline vortioxetine mesylate, hydrogenfumarate, hydrogenmaleate, mesohydrogentartrate, L-(+)-hydrogentartrate, D-(−)-hydrogentartrate, hydrogen sulphate, dihydrogenphosphate and nitrate salt.

WO 2014/177491 discloses Vortioxetine hydrobromide amorphous form and in association with an adsorbent such as Al₂O₃, CaCO₃, MgO, SiO₂, TiO₂ and ZnO.

WO2016/116077 discloses amorphous mixture of Vortioxetine or its hydrobromide salt with a polymer, selected from derivatives of polyacrylates, polymethacrylates, cellulose or polyvinyls.

Though the above-mentioned literature disclose diverse polymorphic forms and processes for the preparation of Vortioxetine or a pharmaceutically acceptable salt, but due to one or more reasons most of them are not particularly convenient and viable to industrial scale-up. Thus, there is an apparent need of a new stable and usable form and its process for preparation, which may be cost effective, industrially viable and may overcome the drawbacks of various prior disclosed processes.

Premixes are characterized by a variety of associated properties such as stability, flow, and solubility. Typical premixes represent a compromise of the above properties, for example, an increase in stability and dissolution properties of the premix. Although there are a variety of premixes, there is a continual search in this field of art for premixes that exhibit an improved mix of properties.

Thus, the instant invention provides a premix in which Vortioxetine hydrobromide exists in a stable crystalline form and a process for manufacturing the premix and pharmaceutical compositions comprising said Vortioxetine hydrobromide premix.

WO 2016/062860 relates to a pharmaceutical composition comprising amorphous Vortioxetine hydrobromide, a process for the preparation thereof, use thereof and a method for stabilizing Vortioxetine hydrobromide in a pharmaceutical composition. However, it has been seen that the amorphous form also gets converted to a stable crystalline form over time. Vortioxetine is also known to be existing in various other crystalline forms. However, the stability of these forms is not established. Hence, the efforts were directed to the development of a stable crystalline form of Vortioxetine with the value added properties of the premix.

The dissolution, bioavailability and other parameters differ as the drug changes from one solid state form to the other. The dissolution and the bioavailability of the crystalline form would be different from that of the amorphous form. These parameters also vary from one crystalline form to the other. In the case of certain water insoluble drugs, it is known that the amorphous form may possess higher intrinsic bioavailability; albeit, this may not be the case universally. Also, it is not desirable to have the drug converted from one solid state form to another over time, as the prediction of the pharmaceutical parameters in such case would not be possible.

In view of the art already available for Vortioxetine, there exists a need for development of a robust process for preparation of an alternate crystalline form, which will in turn be robust and stable.

### OBJECTS OF THE INVENTION

One object of the invention is to provide a Vortioxetine hydrobromide premix in stable form with appropriate dissolution properties and process for preparation thereof.

It is another object of the invention to provide a pharmaceutical composition comprising crystalline Vortioxetine hydrobromide.

A further object of the invention is to provide a process for the preparation of the pharmaceutical composition comprising crystalline Vortioxetine hydrobromide.

Another object of the invention provides a method of stabilizing pharmaceutical composition comprising crystalline Vortioxetine hydrobromide.

### SUMMARY OF THE INVENTION

In accordance with the above objectives, the present invention provides Vortioxetine hydrobromide premix in stable form with appropriate dissolution properties and a process for the preparation thereof. The present invention further provides a pharmaceutical composition comprising crystalline Vortioxetine hydrobromide, a process for preparation thereof and the uses thereof. The invention also provides a method of stabilizing the composition.

### BRIEF DESCRIPTION OF THE DRAWINGS

FIG. 1: FIG. 1 is an X-ray powder diffractogram (XRD) of a Vortioxetine hydrobromide premix according to the present invention.

FIG. 2: FIGS. 2a and 2b show dissolution data of a Vortioxetine hydrobromide premix according to the present invention compared to the beta form of Vortioxetine hydrobromide.

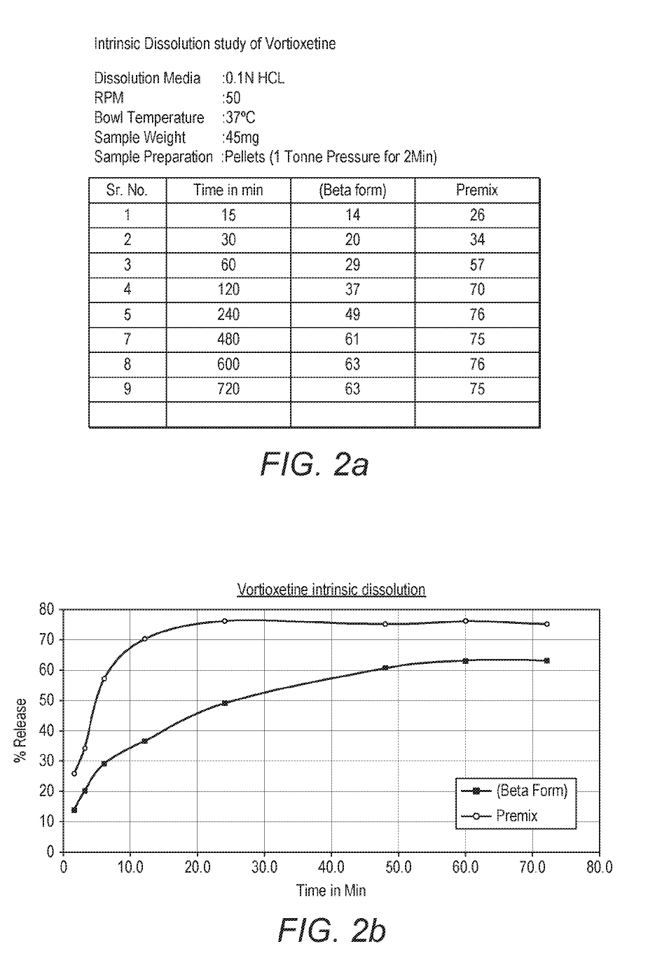

### DETAILED DESCRIPTION OF THE INVENTION

A drug may exist in amorphous and/or crystalline forms. A drug may also exist in more than one or more than one crystalline form, the nature of which may be elucidated by X-ray powder diffraction. The pharmaceutical parameters of a drug differ from one solid state form to another. Some drugs may be more bioavailable in one solid state form than another. In certain cases, it has been seen that the amorphous form of a drug has a lower particle size and hence better bioavailable than a crystalline form of the same drug, albeit, this is not a general rule. In some cases, one crystalline form may be more bioavailable than another.

Another important parameter of any drug used in the formulation of a pharmaceutical composition is stability. Some solid state forms of certain drugs have the tendency to convert into a more stable solid state form over time. Some drugs show a gradual conversion from one form to the other before resting in the final, most stable form. However, such a conversion from one form to the other over a period of time is not desirable. A drug may get converted to a form that may be more bioavailable than the original form, thus potentially leading to toxic side effects. On the contrary, if the drug gets converted to a solid state form that is less bioavailable, it may lead to insufficient drug being available for the treatment of the ailment of concern. Either way, the interconversion of solid state forms is generally not desirable. A composition needs to be formulated such that it comprises the drug in a stable form that does not get converted into another solid state form over a period of time.

WO 2016/062860 relates to a pharmaceutical composition comprising amorphous Vortioxetine hydrobromide, a process for the preparation thereof, use thereof and a method for stabilizing Vortioxetine hydrobromide in a pharmaceutical composition. However, it has been observed that the amorphous form gets converted to a more stable crystalline form.

Thus, there clearly exists a need for formulation of a process that would result in an alternate form of Vortioxetine which is robust and stable.

The term ‘Vortioxetine’ is used in the broad sense to include its pharmaceutically acceptable esters and salts thereof. Vortioxetine may be administered as the free base/free hydroxyl, or it may be administered as an ester or salt. Suitable pharmaceutically acceptable derivatives and/or pharmaceutically acceptable salts include but are not limited to pharmaceutically acceptable solvates, pharmaceutically acceptable hydrates, pharmaceutically acceptable anhydrates, pharmaceutically acceptable enantiomers, pharmaceutically acceptable esters, pharmaceutically acceptable isomers, pharmaceutically acceptable polymorphs, pharmaceutically acceptable tautomers, pharmaceutically acceptable complexes etc.

Pharmaceutically acceptable salts are salts that retain the desired biological activity of the parent compound and do not impart undesirable toxicological effects. Examples of such salts are acid addition salts formed with inorganic acids, for example, hydrochloric, hydrobromic, sulfuric, phosphoric, and nitric acids and the like; salts formed with organic acids such as acetic, oxalic, tartaric, succinic, maleic, fumaric, gluconic, citric, malic, methanesulfonic, p-toluenesulfonic, napthalenesulfonic, and polygalacturonic acids, and the like; salts formed from elemental anions such as chloride, bromide, and iodide; salts formed from metal hydroxides, for example, sodium hydroxide, potassium hydroxide, calcium hydroxide, lithium hydroxide, and magnesium hydroxide; salts formed from metal carbonates, for example, sodium carbonate, potassium carbonate, calcium carbonate, and magnesium carbonate; salts formed from metal bicarbonates, for example, sodium bicarbonate and potassium bicarbonate; salts formed from metal sulfates, for example, sodium sulfate and potassium sulfate; and salts formed from metal nitrates, for example, sodium nitrate and potassium nitrate. Pharmaceutically acceptable and non-pharmaceutically acceptable salts may be prepared using procedures well known in the art, for example, by reacting a sufficiently basic compound such as an amine with a suitable acid comprising a physiologically acceptable anion. Alkali metal (for example, sodium, potassium, or lithium) or alkaline earth metal (for example, calcium) salts of carboxylic acids can also be made. The preferred salt according to the present invention is Vortioxetine hydrobromide. According to one aspect of the present invention, there is provided a premix comprising Vortioxetine hydrobromide in crystalline form and one or more pharmaceutically acceptable polymers.

In one aspect, the present invention provides a Vortioxetine hydrobromide premix having stability and dissolution properties such that it can be easily formulated into pharmaceutically useful composition. Table I below shows that the premix of the present invention advantageously possesses an improved in vitro dissolution rate compared to the beta-crystalline form of Vortioxetine hydrobromide.

TABLE 1

| Sr. No. | Time in min | (Beta Form) | Premix |
|---|---|---|---|
| 1 | 15 | 14 | 26 |
| 2 | 30 | 20 | 34 |
| 3 | 60 | 29 | 57 |
| 4 | 120 | 37 | 70 |
| 5 | 240 | 49 | 76 |
| 7 | 480 | 61 | 75 |
| 8 | 600 | 63 | 76 |
| 9 | 720 | 63 | 75 |

As used herein the term “premix” means two or more components combined to form an admixture. Preferably, the term is used to describe an admixture comprising Vortioxetine hydrobromide and at least one pharmaceutically acceptable polymer.

The present invention provides a vortioxetine hydrobromide premix in stable form with appropriate dissolution properties, wherein Vortioxetine hydrobromide is stabilized by combining with suitable polymers.

further, Vortioxetine hydrobromide is present in stable crystalline form in a Vortioxetine hydrobromide premix. A preferred crystalline form is the beta-crystalline form disclosed in U.S. Pat. No. 8,722,684, the content of which is incorporated herein by reference. The beta form of Vortioxetine hydrobromide is characterized by a melting point of about 231° C. and an X-Ray Powder Diffraction pattern with peaks at 6.89, 9.73, 13.78 and 14.64° 2θ±0.1°. It has an aqueous solubility of about 1.2 mg/ml.

In another aspect of the present invention, there is provided a process for preparing a Vortioxetine hydrobromide premix comprising the steps of
(a) dissolving vortioxetine hydrobromide and a pharmaceutically acceptable polymer in a suitable solvent;
(b) distilling out (removing) the solvent from the solution obtained in step (a); and thereafter
(c) drying the vortioxetine hydrobromide premix so obtained.

The weight ratio of Vortioxetine hydrobromide to polymer may range from about 1:10 to about 10:1. Preferably, the range of Vortioxetine hydrobromide to polymer is about 1:1.

The polymers used in the premix of the present invention and the process for manufacturing the premix are pharmaceutically acceptable. In one aspect, the pharmaceutically acceptable polymer is selected from the group consisting of a cellulose based polymer, an acrylate, a poloxamer, a vinyl homopolymer or copolymer, a polyalkylene glycol (such as polyethylene glycol), an aminosaccharide, polyalkylene oxide (such as polyethylene oxide) and any combination thereof.

Examples of suitable cellulose based polymers include, but are not limited to alkylcelluloses, e.g., methylcellulose; hydroxyalkylcelluloses, e.g., hydroxymethylcellulose, hydroxyethylcellulose (Natrosol™, Ashland, Covington, Ky., hydroxypropylcellulose, hydroxybutylcellulose and weakly substituted hydroxypropylcellulose; hydroxyalkylalkylcelluloses, e.g., ethyl(hydroxyethyl)cellulose, hydroxyethylmethylcellulose and hydroxypropylmethylcellulose (e.g., Methocel, types A, E, K, F, Dow Wolff Cellulosics GmbH, Bomlitz, Germany and Pharmacoat, types 603, 606, 615, 645, Harke Services GmbH, Muelheim an der Ruhr, Germany). Particularly preferred is hydroxypropylmethylcellulose (HPMC) of low viscosity.

Examples of suitable acrylates include polyacrylates including, but not limited to, EUDRAGIT® E PO, methacrylic acid copolymer, polymethacrylates (Eudragit® L-100-55 and Eudragit® E-100, Evonik Degussa Corporation, Parsipanny, N.J.), polyacrylic acid (Carbopol®, The Lubrizol Corporation, Wickliffe, Ohio).

Examples of suitable vinyl homopolymers and copolymers include, but are not limited to, polymers of N-vinylpyrrolidone, in particular povidone, copovidone, polyvinyl alcohol, and polyvinylpyrrolidone (Kollidon™, PVP and PVP-VA, BASF SE, Ludwigshafen, Germany).

Examples of other types of synthetic polymers include, but are not limited to, polyethylene oxide (Polyox™, Dow Chemical Company, Midland, Mich.), polyethyleneglycols of various molecular weights, polyethylene-/polypropylene-/polyethylene-oxide block copolymers and natural gums and polysaccharides-Xanthan gum (Keltrol™, CP Kelco, Atlanta, Ga.), carrageenan, locust bean gum, acacia gum, chitosan, alginic acid, hyaluronic acid, pectin, etc. Suitable polyethyleneglycols are especially Polyethyleneglycol 8000 and Polyethyleneglycol 6000. A suitable polyethylene-/polypropylene-/polyethylene-oxide block copolymer is in particular Pluronic F68.

It is particularly preferred that the pharmaceutically acceptable polymer or co-polymer is selected from the group consisting of a hydroxyalkylcellulose, hydroxyalkylalkylcellulose, preferably HPMC and a polyvinylcaprolactam-polyvinyl acetate-polyethylene glycol graft copolymer. The polyvinylcaprolactam-polyvinyl acetate-polyethylene glycol graft copolymer can for example be obtained from BASF under the trade name Soluplus®.

According to one preferred aspect of the instant invention, EUDRAGIT® E PO is employed as the pharmaceutically acceptable polymer. EUDRAGIT® E PO is a cationic copolymer based on dimethylaminoethyl methacrylate, butyl methacrylate, and methyl methacrylate.this polymer which is available in powder form and is soluble in gastric fluid up to pH 5.0, is swellable and permeable above pH 5.0. Chemically it is known as Poly(butyl methacrylate-co-(2-dimethylaminoethyl) methacrylate-co-methyl methacrylate) 1:2:1.

The process for preparing the Vortioxetine hydrobromide premix comprises dissolving Vortioxetine hydrobromide in a solvent system selected from a group of polar solvents such as C₁-C₄ alcohols or chlorinated organic solvents such as chloroform, dichloromethane, ethylene dichloride alone or in combination. Preferably, the C₁-C₄ alcohol is methanol. Preferably, the chlorinated organic solvent is chloroform.

The dissolution temperatures may range from about 10° C. to about the reflux temperature of the solvent, depending on the solvent used for dissolution, The dissolution temperature may range from about 10° C. to about 120° C. or from about 10° C. to about 80° C., or from about 10° C. to about 65° C. In a preferred aspect, the dissolution temperature is from about 60° C. to about 70° C.

The crystalline nature of Vortioxetine hydrobromide in the premix has been characterized by X-ray diffraction (XRD) as illustrated by I. Powder X-ray Diffraction data were collected on a Rigaku (Smart Lab) X-ray diffractometer using a Cu K-a radiation source under standard operating conditions.

The premix comprising Vortioxetine hydrobromide prepared in accordance with the present invention may advantageously be used to formulate a stable pharmac

One aspect of the invention is directed towards the method of ‘stabilizing’ the composition. As used herein, the term ‘stabilizing’ means that the form of the drug does not undergo conversion to another form within at least 1 week, preferably at least 2 weeks, and more preferably a month. Accordingly, it is most preferred that the drug in the composition prepared as per the invention does not undergo form conversion for at least one month at room temperature.

The present invention provides a composition comprising crystalline Vortioxetine hydrobromide, such that the composition is stable. The composition according to the instant invention comprises a crystalline form of the drug, which crystalline form does not convert to any other solid state form over time. The instant invention further provides a process of preparation of such a pharmaceutical composition and uses thereof. The instant invention further provides method of stabilization of the formulation, such that the form of the drug contained therein is stable and does not get converted to any other solid state form.

As used herein, the term ‘stable’ may relate to chemical and/or physical stability. In one aspect, the term may be taken to mean that the Vortioxetine hydrobromide premix or composition containing it remains in the same solid state as compared to the solid state that it was in when the premix or composition was first prepared. In particular, it may mean that the drug substance contained in the premix or composition containing it does not show a change in the XRPD pattern over at least 7 days, preferably at least one month, and most preferably after at least six months, after preparation.

The term ‘stable’ is also used to mean that the solid state form of Vortioxetine hydrobromide does not show an increase in the percentage of the ‘related substances’ or impurities, when compared to the concentration contained therein, at the time of the preparation of the composition. According to another aspect, the term ‘stable’ means that the dissolution kinetics of the composition does not change over a period of time, as compared to the kinetics, at the time of the preparation of the composition.

The instant invention revolves around the formation of a stable crystalline form of Vortioxetine hydrobromide.

There are a few known form conversion techniques, used to convert the drug substance from one form to another. The most commonly used ones are the use of Cyclodextrin, Hot-Melt extrusion and Solid dispersion techniques.

Cyclodextrins (CDs) are a family of cyclic oligosaccharides with a hydrophilic outer surface and a lipophilic central cavity. These cyclic oligosaccharides consist of (α-1,4)-linked α-D-glucopyranose units. CDs have mainly been used as complexing agents to increase aqueous solubility of poorly soluble drugs and to increase their bioavailability and stability. CDs are used in pharmaceutical applications for numerous purposes, including improving the bioavailability of drugs. The natural α-, β- and γ-cyclodextrin consist of six, seven, and eight glucopyranose units, respectively. Cyclodextrin derivatives of pharmaceutical interest include the hydroxypropyl derivatives of β- and γ-cyclodextrin, the randomly methylated β-cyclodextrin, sulfobutylether β-cyclodextrin, and the so-called branched cyclodextrins such as glucosyl-β-cyclodextrin. However, the CDs are too large, resulting in the increase of the tablet weight, and hence not the technique of choice.

The other method used is the Hot-Melt extrusion (HME) technique. HME is a continuous pharmaceutical process involving pumping the polymeric materials with a rotating screw at temperatures above their glass transition temperature (T_{g}) and sometimes above the melting temperature (Tₘ) to achieve molecular level mixing of the active compounds and the other ingredients. This molecular mixing converts the components into an amorphous product with a uniform shape and density, thereby increasing the dissolution profile of the poorly water-soluble drug. Polymers are the most important excipients in the HME formulations. Molten or softened polymers act as binders for granulations, thus requiring no solvents. Mixing occurs thoroughly in the molten state and the drug is embedded in the polymeric matrix. Commonly used polymers are Polyethylene glycol, Polyethylene oxide, Hydroxypropyl cellulose (Klucel®), Ethyl cellulose, Hydroxypropylmethyl cellulose, Poly(dimethylamino ethyl methacrylate-co-methacrylate ester), Ammonio-comethacrylate copolymer, Poly(vinyl pyrrolidone)-(Kollidon®) and Poly(vinyl acetate).

However, this technique is not desirable in cases where the melting point of the drug to be converted is very high. For the process to work, it is required that the drug undergoes complete melting, which would lead to complete conversion. The melting point of the drug Vortioxetine is very high, in order of 227-230 degrees. The normal temperatures that are employed in the laboratory is around 180 C, maximizing to 200 C. The HME in this case was attempted with Klucel and Kollidone VA 64. In both the cases, it resulted in the formation of the amorphous form with slight crystalline peaks. On charging this form on stability, it was observed that there was increase in the intensity of the crystalline peaks, indicating that the resulting amorphous form gets converted to the crystalline form, thus indicating instability of the product. This could be attributed to the incomplete melting of the drug and thus incomplete form conversion.

Solid dispersion is a simple two component system where the drug and the polymer act as solute and solvent, respectively. In particular, the drug is dispersed in at least one carrier. When the said dispersion of the components is such that the system is chemically and physically uniform or homogenous throughout or consists of only one thermodynamic phase, it is called a ‘solid solution’. A ‘solid solution’ can also be considered as a system in a solid state wherein the drug is molecularly dispersed within at least one carrier.

Various FDA approved medicines use the solid dispersion technologies.

At least the use of PVP, PVP/VA, HPMC and HPMCAS have been successfully used in the commercial production of these formulations. WO 2016/062860 relates to the use of solid dispersion to result in the formation of amorphous Vortioxetine hydrobromide. However, as highlighted earlier, the amorphous form is found to be unstable and gets converted to the crystalline form. This conversion of the form over time is undesirable for the reasons mentioned earlier.

As used herein, the term ‘crystalline’ means a solid body having a uniform crystalline order that can be detected, measured and monitored, e.g., by X-ray diffraction, FT-Raman Spectroscopy and Differential Scanning calorimetry (DSC).

As used herein, the term ‘crystalline Vortioxetine hydrobromide’ means Vortioxetine hydrobromide contained in solid solution or solid dispersion in crystalline state, wherein at least 95% represents crystalline state of Vortioxetine hydrobromide in the solid solution or the solid dispersion, preferably, 98%, more preferably 99%, and most preferably 100%. According to one aspect, the crystallinity of Vortioxetine hydrobromide prepared as per the instant invention results in no detectable amorphous structure of the drug, when analysed by the routine techniques used for the purpose.

According to another aspect, the carrier used for the preparation of the crystalline Vortioxetine hydrobromide, as per the instant invention, is an organic polymer or co-polymer.

The polymer may be a cellulose based polymer, acrylate, poloxamer, vinyl homopolymer or copolymer, polyethylene glycol, aminosaccharide or polyethylene oxide.

Examples of a cellulose based polymer include, but are not limited to alkylcelluloses, e.g., methylcellulose; hydroxyalkylcelluloses, e.g., hydroxymethylcellulose, hydroxyethylcellulose (Natrosol™, Ashland, Covington, Ky.), hydroxypropylcellulose, hydroxybutylcellulose and weakly substituted hydroxypropylcellulose; hydroxyalkylalkylcelluloses, e.g., ethyl(hydroxyethyl)cellulose, hydroxyethylmethylcellulose and hydroxypropylmethylcellulose (e.g., Methocel, types A, E, K, F, Dow Wolff Cellulosics GmbH, Bomlitz, Germany and Pharmacoat, types 603, 606, 615, 645, Harke Services GmbH, Muelheim an der Ruhr, Germany). Particularly preferred is HPMC of low viscosity. Examples of acrylate include polyacrylates including, but are not limited to, EUDRAGIT® E PO, methacrylic acid copolymer, polymethacrylates (Eudragit® L-100-55 and Eudragit® E-100, Evonik Degussa Corporation, Parsipanny, N.J.), polyacrylic acid (Carbopol®, The Lubrizol Corporation, Wickliffe, Ohio).

Examples of vinyl homopolymers and copolymers include, but are not limited to, polymers of N-vinylpyrrolidone, in particular povidone, copovidone, polyvinyl alcohol, and polyvinylpyrrolidone (Kollidon™, PVP and PVP-VA, BASF SE, Ludwigshafen, Germany).

Examples of other types of synthetic polymers include, but are not limited to, polyethylene oxide (Polyox™, Dow Chemical Company, Midland, Mich.), polyethyleneglycols of various molecular weights, polyethylene-/polypropylene-/polyethylene-oxide block copolymers and natural gums and polysaccharides-Xanthan gum (Keltrol™, CP Kelco, Atlanta, Ga.), carrageenan, locust bean gum, acacia gum, chitosan, alginic acid, hyaluronic acid, pectin, etc. Suitable polyethyleneglycols are especially Polyethyleneglycol 8000 and Polyethyleneglycol 6000. A suitable polyethylene-/polypropylene-/polyethylene-oxide block copolymer is in particular Pluronic F68.

It is particularly preferred that the organic polymer or co-polymer is selected from the list consisting of a hydroxyalkylcellulose, hydroxyalkylalkylcellulose, preferably hydroxypropylmethylcellulose (HPMC) and a polyvinylcaprolactam-polyvinyl acetate-polyethylene glycol graft copolymer. The polyvinylcaprolactam-polyvinyl acetate-polyethylene glycol graft copolymer can for example be obtained from BASF under the trade name Soluplus®.

According to one preferred aspect of the instant invention, EUDRAGIT® E PO was employed as the polymer for the preparation of the solid dispersion. EUDRAGIT® E PO is a cationic copolymer based on dimethylaminoethyl methacrylate, butyl methacrylate, and methyl methacrylate.this polymer which is available in powder form and is Soluble in gastric fluid up to pH 5.0, is swellable and permeable above pH 5.0. Chemically it is known as Poly(butyl methacrylate-co-(2-dimethylaminoethyl) methacrylate-co-methyl methacrylate) 1:2:1.

For the preparation of a solid dispersion of a drug, it is also important to maintain the drug: polymer ratio. This ratio becomes critical in view of obtainment of the consistent stable form each time. The robustness and the stability of the form of the drug obtained is the function of the drug: polymer ratio employed and is very critical. A slight variation in this ratio may lead to form conversion and/or formation of undesirable form of the drug.

The drug: polymer ratio for the preparation of the solid dispersion may be from about 0.1:10 to about 10:0.1. According to one preferred aspect of the invention, the amount of the drug is more as compared to the polymer used. According to another preferred aspect, the amount of the polymer is more than the drug used to prepare the solid dispersion. The said ratio, according to a more preferred aspect is from about 0.1:1 to about 1:0.1, most preferably about 1:1.

One aspect of the instant invention provides the process of preparing a pharmaceutical composition comprising crystalline Vortioxetine hydrobromide. The drug is dissolved in a suitable solvent. In another aspect, the drug is dissolved in a combination of more than one solvent to form a drug solution. The solvent used could be a protic solvent, an aprotic solvent, or a mixture of a protic solvent and an aprotic solvent. Examples for the solvents that may be employed are alcohols, aliphatic hydrocarbons or esters. Particularly preferred solvents are methanol, ethanol, dichloromethane, isopropanol and acetone. Water can also act as the solvent for dissolving the drug, as per the instant invention. In case a combination of more than one solvent is used, the ratio of the different solvents used may become important. The polymer is added to the said drug solution and granulation is carried out using one of the various known techniques. According to one preferable aspect of the invention, the granulation may be carried out in a fluidized bed using an appropriate carrier. One of the other aspects of the invention also relates to the use of wet granulation techniques, such as high shear granulation, or the like. The granules may then be blended with extra granular materials and lubricated. The blend obtained thus, may be packaged in a sachet, filled in a capsule or compressed to form a tablet. The tablets may be optionally coated.

The drug may be mixed with a mixture of ethanol and methylene dichloride (MDC) to obtain a drug solution. Preferably, the ratio of ethanol:MDC used is in the range from about 0.1:10 to about 10:0.1, more preferably from about 0.5:5 to about 5:0.5 and most preferably about 1:3. Eudragit EP O may preferably be mixed with the said drug solution to obtain a drug premix. The premix may then be sprayed in a fluidized bed reactor on a binder or a carrier. The binders or the carriers known in the art may be used for the purpose. Microcrystalline cellulose is the carrier or the absorbent that may be preferably employed for the purpose of preparing the composition as per the instant invention.

The spray rate of the premix may be of utmost importance in final outcome as regards the nature, form and the stability of the end product. Slower spray rate combined with high atomization may lead to faster rate of evaporation, whereas the faster spray rate, combined with lesser atomization, may lead to slower rate of evaporation.

A faster rate of evaporation results in the formation of amorphous drug product, which as seen earlier, is unstable and has the tendency to convert into crystalline form. A slower rate of evaporation results in the formation of the crystalline drug product, as per the instant invention. The spray rate may be dependent on the batch size that is being manufactured. According to one aspect of the present invention, the batch size may be from about 1000 to 10,000 tablets. In a preferred aspect, the batch size may be between 2000 and 5000 tablets.

The spray rate from 0.1 g/min up to about 6 g/min of the premix may result in the formation of amorphous form., The spray rate up from about 6 g/min, up to about 25 g/min of the premix, may result in the formation of the crystalline form as per the instant invention. According to a preferred aspect, the spray rate used may be between 7 g/min and 10 g/min, so as to get the form of the drug product as per the instant invention.

The values of the spray rate may change as per the batch size. The batch size may vary significantly. For a larger batch, for example about 150,000 tablets, according to one aspect of the invention, the optimum spray rate may fall within a range of from about 50 to about 500 g/min.

As used herein, the term ‘about’ means up to 10% change in the values on the higher as well as the lower side of the value.

The degree of atomization of the air may specifically play a part in the formation of one form over the other. The higher or more degree of atomization of the air may lead to formation of amorphous form, whereas the lower or lesser degree of atomization of air may lead specifically to the formation of the crystalline form as per the instant invention. Other factors that may specifically affect the formation of the drug product as per the instant invention may be the fluidization process used for the granulation. The other factor that may affect is the obtainment of the form as per the instant invention is the distance between the spray gun and the powder bed.

At least one of the above-mentioned factors may affect the formation of the drug product as per the instant invention.

In some aspect of the invention, the term ‘drug’ and ‘drug product’ may be used interchangeably. However, in at least one aspect, the meaning of the terms would differ.

The granules may be dried before the extra granular ingredients are added to make a blend, to achieve an appropriate loss on drying (LOD). The drying of the granules may be of particular importance in the final outcome as regards the nature, form and the stability of the end product. The rate and the temperature used for the drying of the granules may result in crystalline or amorphous form of the drug. According to one aspect of the invention, the rate of drying and the temperature is so used that the result is formation of crystalline Vortioxetine hydrobromide.

According to the present invention, the drug may be present in an amount of from about 1 to about 90% (w/w) based on the total weight of the solid solution or solid dispersion, preferably from about 10 to about 70% (w/w), more preferably from about 15 to about 60% (w/w), and most preferably from about 20 to about 40% (w/w).

According to another aspect of the invention, the solid solution or solid dispersion is present in an amount of from about 1 to about 100% (w/w) based on the total weight of the pharmaceutical composition. In a preferred aspect, the solid solution or solid dispersion is present in an amount of from about 10 to about 90% (w/w), preferably from about 20 to about 80% (w/w), more preferably from about 40 to about 80% (w/w), and most preferably from about 60 to about 80% (w/w). According to one other aspect of the invention, the drug is present in an amount of from about 1 to about 60% (w/w) based on the total weight of the pharmaceutical composition. In a preferred aspect, the drug is present in an amount of from about 2 to about 50% (w/w) based on the total weight of the pharmaceutical composition, preferably from about 5 to about 40% (w/w), more preferably from about 8 to about 30% (w/w), and most preferably from about 10 to about 20% (w/w).

Preferably, the composition of the instant invention may be provided in the form of a pharmaceutical composition such as but not limited to, unit dosage forms including tablets, capsules (filled with powders, pellets, beads, mini-tablets, pills, micro-pellets, small tablet units, multiple unit pellet systems (MUPS), disintegrating tablets, dispersible tablets, granules, and microspheres, multiparticulates), sachets (filled with powders, pellets, beads, mini-tablets, pills, micro-pellets, small tablet units, MUPS, disintegrating tablets, dispersible tablets, granules, and microspheres, multiparticulates), powders for reconstitution, transdermal patches and sprinkles, other dosage forms such as controlled release formulations, lyophilized formulations, modified release formulations, delayed release formulations, extended release formulations, pulsatile release formulations, dual release formulations and the like. Liquid or semisolid dosage form (liquids, suspensions, solutions, dispersions, ointments, creams, emulsions, microemulsions, sprays, patches, spot-on), injection preparations, parenteral, topical, inhalations, buccal, nasal etc. may also be envisaged under the ambit of the invention.

The pharmaceutical composition of the present invention can consist exclusively of the solid solution or solid dispersion and in a preferred aspect the pharmaceutical composition comprises only the solid solution or solid dispersion as defined above.

The solid solution or solid dispersion of the present invention can consist exclusively of Vortioxetine hydrobromide and the at least one organic carrier. However, in a further preferred aspect the solid solution or solid dispersion of the present invention contains Vortioxetine hydrobromide, the at least one organic carrier and at least one further ingredient. In general, the further ingredient is contained in an amount of about 0.01 to about 80%, preferably of about 5 to about 50% by weight relative to the weight of the solid solution or solid dispersion.

Suitable excipients may be used for formulating the dosage forms according to the present invention such as, but not limited to, stabilizers or surfactants, surface modifiers, wetting agents, suspending agents, isotonizing agents, chelating agents, osmolality adjusters, pH adjusters, emulsifiers, viscosity modifying agents, polymers including extended release polymers, stabilizers, disintegrants or super disintegrants, diluents, plasticizers, binders, glidants, lubricants, sweeteners, flavoring agents, anti-caking agents, opacifiers, anti-microbial agents, antifoaming agents, emulsifiers, buffering agents, coloring agents, carriers, fillers, anti-adherents, solvents, taste-masking agents, preservatives, antioxidants, texture enhancers, channeling agents, coating agents or combinations thereof. Certain specific excipients may be used for specific dosage forms, as per the requirement of the invention.

One of ordinary skill in the art may select one or more of the aforementioned excipients with respect to the particular desired properties of the granulate and/or solid oral dosage form by routine experimentation and without any undue burden. The amount of each excipient used may vary within ranges conventional in the art. The following references disclose techniques and excipients used to formulate oral dosage forms (see The Handbook of Pharmaceutical Excipients, 4th edition, Rowe et al., Eds., American Pharmaceuticals Association (2003); and Remington: the Science and Practice of Pharmacy, 20th edition, Gennaro, Ed., Lippincott Williams & Wilkins (2000).

Typical excipients include antioxidants. Antioxidants may be used to protect ingredients of the composition from oxidizing agents that are included within or come in contact with the composition. Examples of antioxidants include water soluble antioxidants such as ascorbic acid, sodium sulfite, metabisulfite, sodium miosulfite, sodium formaldehyde, sulfoxylate, isoascorbic acid, isoascorbic acid, cysteine hydrochloride, 1 ,4-diazobicyclo-(2,2,2)-octane, and mixtures thereof. Examples of oil-soluble antioxidants include ascorbyl palmitate, butylated hydroxyanisole, butylated hydroxytoluene, potassium propyl gallate, octyl gallate, dodecyl gallate, phenyl-a-napthyl-amine, and tocopherols such as a-tocopherol.

Examples of binders include, but are not limited to, starches, celluloses and derivatives thereof, sucrose, dextrose, corn syrup, polysaccharides, and gelatin. Examples of celluloses and derivatives thereof include for example, microcrystalline cellulose, e.g., AVICEL PH from FMC (Philadelphia, Pa.). Particularly preferred is microcrystalline cellulose, e.g., AVICEL PH 200 from FMC (Philadelphia, Pa.).

Examples of bulking agents include, without limitation, PEGs, mannitol, trehalose, lactose, sucrose, sucrose, glycine, cyclodextrins, dextran and derivatives and mixtures thereof. Especially preferred is mannitol, e.g. PEARLITOL®50C from Roquette Pharma (Lestrem, France). Bulking agents are ingredients which may provide bulk to a pharmaceutical composition. Examples of bulking agents include, without limitation, PEGs, mannitol, trehalose, lactose, sucrose, polyvinyl pyrrolidone, sucrose, glycine, cyclodextrins, dextran and derivatives and mixtures thereof. Especially preferred is mannitol, e.g. PEARLITOL® 50C from Roquette Pharma (Lestrem, France).

Examples of absorbents include, without limitation, MCC, DCP, Magnesium aluminium silicate, silicon di-oxide (Syloid), Colloidal silicon dioxide (Aerosil, Cab-o-sil), Al₂O₃, CaCO₃, MgO, SiO₂, TiO₂ and ZnO

Examples of disintegrants include, but are not limited to starches, e.g. sodium carboxymethyl starch or sodium starch glycolate; clays; alginates; gums; cross-linked polymers, e.g., cross- linked polyvinyl pyrrolidone or crospovidone, e.g., POLYPLASDONE XL from International Specialty Products (Wayne, N.J.); cross-linked sodium carboxymethylcellulose or croscarmellose sodium, e.g., AC-DI-SOL from FMC; and cross-linked calcium carboxymethylcellulose; soy polysaccharides; and guar gum. Especially preferred is sodium starch glycolate, e.g. PRIMOJEL®from DFE-Pharma (Goch, Germany), ion exchange resins.

Examples of pharmaceutically fillers include, but are not limited to confectioner's sugar, compressible sugar, dextrates, dextrin, dextrose, lactose, mannitol, microcrystalline cellulose, powdered cellulose, sorbitol, sucrose and talc.

Examples of pharmaceutically acceptable glidants and pharmaceutically acceptable lubricants include, but are not limited to, colloidal silica, magnesium trisilicate, starches, talc, tribasic calcium phosphate, magnesium stearate, aluminum stearate, calcium stearate, magnesium carbonate, magnesium oxide, polyethylene glycol, powdered cellulose and microcrystalline cellulose. Typically, a lubricant may be present in an amount from about 0.1% to about 5% by weight of the composition; whereas, the glidant, e.g., may be present in an amount from about 0.1% to about 10% by weight. Especially preferred is magnesium stearate.

Preservatives may also be used to protect the composition from degradation and/or microbial contamination. Examples of preservatives include liquipar oil, phenoxyethanol, methyl paraben, propyl paraben, butyl paraben, isopropyl paraben, isobutyl paraben, diazolidinyl urea, imidazolidinyl urea, diazolindyl urea, benzalkonium chloride, benzethonium chloride, phenol, and mixtures thereof (e.g., liquipar oil).

Surfactants include, but are not limited to, fatty acid and alkyl sulfonates; benzethonium chloride, e.g., HYAMINE 1622 from Lonza, Inc. (Fairlawn, N.J.); polyoxyethylene sorbitan fatty acid esters, e.g., the TWEEN Series from Uniqema (Wilmington, Del.); and natural surfactants, such as sodium taurocholic acid, 1-palmitoyl-2-Sn-glycero-3-phosphocholine, lecithin and other phospholipids, Span, Cremophor. It is preferred that the solid dispersion or solid solution contains microcrystalline cellulose.

The organic carrier and “optional further ingredients” differ in that the organic carrier forms a continuous phase in which the active ingredient and the optional further ingredients are dispersed or dissolved.

Buffering agents may be used to maintain an established pH of the composition. Examples of buffering agents included sodium citrate, calcium acetate, potassium metaphosphate, potassium phosphate monobasic, and tartaric acid.

Examples of pharmaceutically acceptable diluents and pharmaceutically acceptable fillers include, but are not limited to, confectioner's sugar, compressible sugar, dextrates, dextrin, dextrose, lactose, mannitol, microcrystalline cellulose, powdered cellulose, sorbitol, sucrose and talc.

Surfactants are agents used to stabilize multi-phasic compositions, e.g., used as wetting agents, antifoam agents, emulsifiers, dispersing agents, and penetrants. Surfactants include, but are not limited to, fatty acid and alkyl sulfonates; benzethonium chloride, e.g., HYAMINE 1622 from Lonza, Inc. (Fairlawn, N.J.); polyoxyethylene sorbitan fatty acid esters, e.g., the TWEEN Series from Uniqema (Wilmington, Del.); and natural surfactants, such as sodium taurocholic acid, 1 -palmitoyl-2-Sn-glycero-3-phosphocholine, lecithin and other phospholipids. Such surfactants, e.g., minimize aggregation of lyophilized particles during reconstitution of the product. Surfactants, if present, are typically used in an amount of from about 0.01% to about 5% w/w.

A cosurfactant is a surface-active agent that acts in addition to the surfactant by further lowering the interfacial energy but that cannot form micellar aggregates by itself. Cosurfactants can be, for example, hydrophilic or lipophilic. Examples of a cosurfactant include, but are not limited to, cetyl alcohol and stearyl alcohol.

According to one preferred aspect, one or more of Mannitol, Microcrystalline cellulose, Magnesium Aluminometasilicate (Neusilin US2) and Sodium Starch Glycolate may be used as the extra granular ingredients.

The pharmaceutical composition as per the instant invention may be used in the treatment of a disease selected from affective disorders, depression, major depressive disorder, postnatal depression, depression associated with bipolar disorder, Alzheimer's disease, psychosis, cancer, age or Parkinson's disease, anxiety, general anxiety disorder, social anxiety disorder, obsessive compulsive disorder, panic disorder, panic attacks, phobia, social phobia, agoraphobia, stress urinary incontinence, emesis, irritable bowel syndrome, eating disorders, chronic pain, partial responders, treatment resistant depression, Alzheimer's disease, cognitive impairment, attention deficit hyperactivity disorder, melancholia, posttraumatic stress disorder, hot flushes, sleep apnea, alcohol, nicotine or carbohydrate craving, substance abuse and alcohol or drug abuse. For the above-mentioned indications, the appropriate dosage will vary depending on, for example, the host, the mode of administration, the nature and severity of the condition, disease or disorder or the effect desired.

The drug may be conveniently administered in a unit dose form comprising from about 1 to about 50 mg of Vortioxetine or a pharmaceutically acceptable derivative thereof, such as Vortioxetine hydrobromide. The total daily dose is expected to be in the range of from about 1 to about 20 mg of Vortioxetine or a pharmaceutically acceptable derivative thereof Vortioxetine hydrobromide.

In another aspect of the present invention there is provided a premix comprising Vortioxetine hydrobromide and one or more pharmaceutically acceptable polymers for use in medicine, preferably for the treatment, prophylaxis or management of major depressive episodes in adults.

In another aspect of the present invention there is provided the use of a premix comprising Vortioxetine hydrobromide and one or more pharmaceutically acceptable polymers in the manufacture of a medicament for the treatment, prophylaxis or management of major depressive episodes in adults.

In order that this invention is more fully understood, the following preparative and testing methods and examples are set forth. These methods are for the purpose of illustration only and are not to be construed as limiting the scope of the invention in any way.

Table 2: The following table gives the various analytical parameters for the formulation prepared according to the instant invention.

TABLE 2

| Batch No. | Test |
|---|---|
| Strength | 20 mg |
| Content uniformity (%) | 104.0 (7.1) |
| (AV) | |
| Assay (%) | 105.5 |
| RS (related substances) | |
| (Initial) (%) | |
| Sulphoxide impurity | <0.1 |
| SMI (single max impurity) | 0.29 |
| Total (impurity) | 0.29 |
| Water content (%) | 5.20 |

The above shows that the formulation obtained as per the instant invention passes the United States Pharmacopeia (USP) criteria for assay and impurity levels.

Table 3: Gives the comparison of the dissolution of the formulation prepared as per the instant invention as compared to the Reference Listed Drug (RLD) (Trintellix®, (earlier Brintellix®)). (Media used was USP type II/Paddle/900 ml pH 6.8/50 rpm/45 min)

TABLE 3

| Time Points | RLD | Test |
|---|---|---|
| 10 | 18 | 14 |
| 15 | 22 | 24 |
| 20 | 27 | 32 |
| 30 | 34 | 40 |
| 45 | 45 | 55 |

The dissolution for the test formulation prepared as per the instant invention is comparable to the RLD.

### EXAMPLES

### Example 1

Charged 100 g of vortioxetine hydrobromide (HBr) at 30° C. to 100 g Eudragit E PO, 2000 ml methanol. The reaction mixture was stirred to get clear solution at 30-35° C. The solvent was distilled under vacuum at 35-40° C. (Outer temperature) to get solid premix. The premix was dried at 50° C. under vacuum for 4.0 hrs. Yield=190 g.

### Example 2

Charged 106 g of vortioxetine hydrobromide and 94 g Eudragit E PO, 1600 ml methanol. The reaction mixture was stirred to get clear solution at 30-35° C. The solvent was distilled under vacuum at 35-40° C. (Outer temperature) to get solid premix. The premix was dried at 50° C. under vacuum for 4.0 hrs. Yield=198 g.

### Example 3

Charged 100 g of vortioxetine hydrobromide and 150 g Eudragit E PO, 1800 ml methanol. The reaction mixture was stirred to get clear solution at 30-35° C. The solvent was distilled under vacuum at 35-40° C. (Outer temperature) to get solid premix. The premix was dried at 50° C. under vacuum for 4.0hrs. Yield=242 g.

### Example 4

Charged 5.0 g of vortioxetine hydrobromide and 5.0 g Eudragit E PO, 25 ml ethanol and 25 ml MDC. The reaction mixture was stirred to get clear solution at 25-30° C. The solvent was distilled under vacuum at 40-45° C. (Outer temperature) to get solid premix. Yield=10 g.

### Example 5

Charged 5.0 g of vortioxetine hydrobromide and 5.0 g Eudragit E PO, 25 ml ethanol and 25 ml MDC. The reaction mixture was stirred to get clear solution at 25-30° C. The solvent was distilled under vacuum at 30° C. (Outer temperature) to get solid premix. Yield=10 g.

### Example 6

Charged 100 g of vortioxetine hydrobromide and 100 g Eudragit E PO, 1800 ml methanol. The reaction mixture was stirred to get clear solution at 30-35° C. The solvent was distilled under vacuum at 35-40° C. (Outer temperature) to get solid premix. The premix was dried at 50° C. under vacuum for 4.0 hrs. Yield=190 g.

### Example 7

The following example gives the process of formulating pharmaceutical composition as per the instant invention
1. Dissolve Vortioxetine Hydrobromide and Eudragit EPO in Ethanol: methylene dichloride (MDC) mixture.
2. Granulate in Fluidized bed reactor using Microcrystalline cellulose as a carrier.
3. Blend with extragranular materials
4. Lubricate using magnesium stearate.
5. Compress the lubricated blend into tablets and coat

### Example 8

The following example gives the formulation of the composition obtained as per the instant invention

| Ingredients | Qty (mg/tab) |
|---|---|
| Dry Mix: | |
| Vortioxetine HBr | 25.42 |
| Microcrystalline Cellulose | 70.16 |
| Aminomethacrylate Copolymer | 25.42 |
| Ethanol | q.s. |
| Methylene dichloride | q.s. |
| Total weight | 121.00 |
| Extragranular part | |
| Mannitol | 22.50 |
| Sodium Starch glycolate | 5.00 |
| Aerosil | 4.50 |
| Magnesium Stearate | 2.00 |
| Total weight | 155.00 |
| Opadry | 5.00 |
| Total weight | 160.00 |

1. A premix comprising Vortioxetine hydrobromide and at least one pharmaceutically acceptable polymer.

2. The premix according to claim 1, wherein the premix is crystalline.

3. The premix according to claim 1, wherein the Vortioxetine hydrobromide is present in crystalline form.

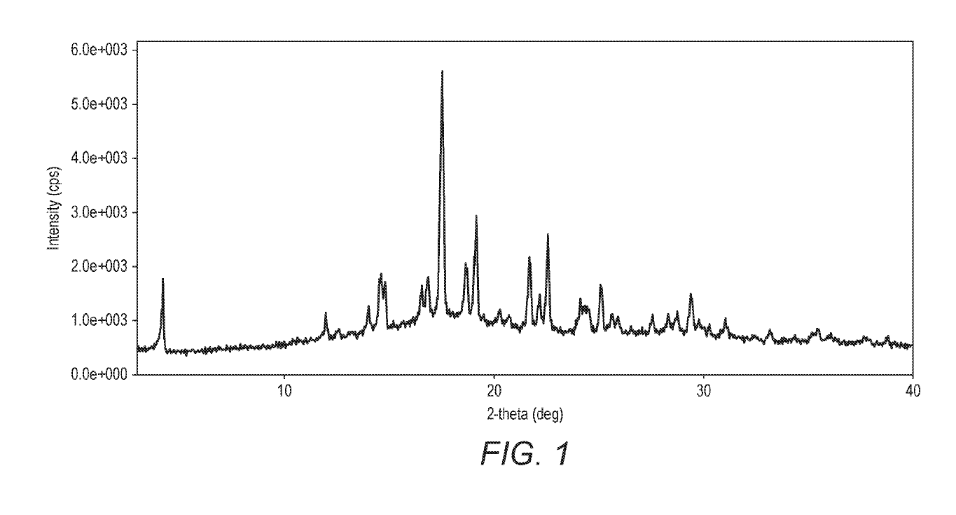

4. The premix according to claim 2, having an XRD pattern as shown in FIG. 1.

5. The premix according to claim 1, wherein the pharmaceutically acceptable polymer is selected from the group consisting of a cellulose based polymer, an acrylate, a poloxamer, a vinyl homopolymer or copolymer, a polyalkylene glycol such as polyethylene glycol, an aminosaccharide, a polyalkylene oxide such as polyethylene oxide, and any combination thereof.

6. The premix according to claim 5, wherein the pharmaceutically acceptable polymer is a cellulose based polymer.

7. The premix according to claim 6, wherein the cellulose based polymer is selected from the group consisting of alkylcelluloses, hydroxyalkylcelluloses, hydroxyalkylalkylcelluloses, and any combination thereof.

8. The premix according to claim 5, wherein the pharmaceutically acceptable polymer is an acrylate polymer.

9. The premix according to claim 8, wherein the acrylate polymer is selected from the group consisting of EUDRAGIT® EP O, a methacrylic acid copolymer, a polymethacrylate and a polyacrylic acid, and any combination thereof.

10. The premix according to claim 5, wherein the pharmaceutically acceptable polymer is a vinyl homopolymer or copolymer.

11. The premix according to claim 10, wherein the vinyl homopolymer or copolymer is selected from the group consisting of povidone, copovidone, polyvinyl alcohol and polyvinylpyrrolidone, and any combination thereof.

12. The premix according to claim 5, wherein the pharmaceutically acceptable polymer is selected from the group consisting of a hydroxyalkylcellulose, a hydroxyalkylalkylcellulose and a polyvinylcaprolactam-polyvinyl acetate-polyethylene glycol graft copolymer, and any combination thereof.

13. The premix according to claim 5, wherein the pharmaceutically acceptable polymer is EUDRAGIT® E PO.

14. The premix according to claim 1, wherein the weight ratio of Vortioxetine hydrobromide to pharmaceutically acceptable polymer is from about 1:10 to about 10:1.

15. The premix according to claim 14, wherein the weight ratio of Vortioxetine hydrobromide to pharmaceutically acceptable polymer is about 1:1.

16. The process for preparing a premix of Vortioxetine hydrobromide according to claim 1 comprising the steps of:
(a) dissolving Vortioxetine hydrobromide and at least one pharmaceutically acceptable polymer in at least one suitable solvent;
(b) distilling out the solvent from the solution obtained in step (a); and thereafter
(c) drying the Vortioxetine hydrobromide premix so obtained.

17. The process according to claim 16, wherein the pharmaceutically acceptable polymer is selected from the group consisting of a cellulose based polymer, an acrylate, a poloxamer, a vinyl homopolymer or copolymer, a polyalkylene glycol such as polyethylene glycol, an aminosaccharide, a polyalkylene oxide such as polyethylene oxide, and any combination thereof.

18. The process according to claim 16, wherein the pharmaceutically acceptable polymer is selected from the group consisting of hydroxyalkylcellulose, a hydroxyalkylalkylcelluloseand and a polyvinylcaprolactam-polyvinyl acetate-polyethylene glycol graft copolymer, and any combination thereof.

19. The process according to claim 16, wherein the pharmaceutically acceptable polymer is EUDRAGIT® EP O.

20. The process according to claim 16, wherein the solvent is a C₁-C₄ alcohol, a chlorinated organic solvent or any combination thereof.

21. The process according to claim 20, wherein the solvent is methanol, ethanol, chloroform, dichloromethane and ethylene dichloride or any combination thereof.

22. The process according to claim 20, wherein the chlorinated organic solvent is chloroform.

23. The process according to claim 16, wherein the dissolution temperature in step (a) ranges from about 10° C. to about 120° C.

24. A pharmaceutical composition comprising a premix according to claim 1 and one or more pharmaceutically acceptable excipients.

25. (canceled)

26. A pharmaceutical composition comprising Vortioxetine hydrobromide having an XRD pattern as shown in FIG. 1 and, optionally, one or more pharmaceutically acceptable excipients.

27. The process for preparing a pharmaceutical composition according to claim 24, comprising the steps of:
(i) preparing a Vortioxetine hydrobromide premix comprising the steps of:
(a) dissolving Vortioxetine hydrobromide and at least one pharmaceutically acceptable polymer in at least one suitable solvent;
(b) distilling out the solvent from the solution obtained in step (a) and thereafter
(c) drying the Vortioxetine hydrobromide premix to obtained;
(ii) granulating the premix with a suitable carrier material;
(iii) blending the resulting granules with one or more extragranular materials; and
(iv) compressing the resulting mixture to form a pharmaceutical composition.

28. The premix obtainable by a process according to claim 16.

29. (canceled)
